(19) United States
(12) Patent Application Publication
(10) Pub. No.: US 2025/0035768 A1
(43) Pub. Date: Jan. 30, 2025

(54) FREQUENCY MODULATED CONTINUOUS WAVE RADAR AND DETECTION METHOD THEREOF

(71) Applicant: Wistron Corp., New Taipei City (TW)

(72) Inventors: Yao Tsung CHANG, New Taipei City (TW); Chuan Yen KAO, New Taipei City (TW)

(21) Appl. No.: 18/452,708

(22) Filed: Aug. 21, 2023

(30) Foreign Application Priority Data
Jul. 27, 2023 (TW) 112128065

(51) Int. Cl. G01S 13/536; G01S 7/35; G01S 13/56

(52) U.S. Cl. CPC G01S 13/536; G01S 7/354; G01S 7/356; G01S 13/56

(57) ABSTRACT

An FMCW (Frequency Modulated Continuous Wave) radar and a detection method thereof are provided. The FMCW radar for detecting an object with a sensor includes a chirp signal generator, a transmitter module, a receiver module, a processing module, and a computing module. The chirp signal generator generates a radar signal. The transmitter module transmits the radar signal to the object. The receiver module receives a reflection signal from the object. The reflection signal includes a feature signal and a sense signal. The processing module generates an integrated digital signal according to the reflection signal and the radar signal. The computing module analyzes the integrated digital signal, so as to obtain first digital information and second digital information. The first digital information corresponds to the feature signal. The second digital information corresponds to the sense signal.

### CROSS REFERENCE TO RELATED APPLICATIONS

This application claims priority of Taiwan Patent Application No. 112128065 filed on Jul. 27, 2023, the entirety of which is incorporated by reference herein.

### BACKGROUND OF THE INVENTION

### Field of the Invention

The invention relates to an FMCW (Frequency Modulated Continuous Wave) radar, and more particularly, to an FMCW radar and a detection method thereof.

### Description of the Related Art

In recent years, technology to detect physiological information (Vital Signs) has seen rapid development (e.g., body temperature measurement, blood oxygen concentration detection, and non-contact radar detection devices for heartbeat and respiratory rates). These have been gradually applied in patient care, long-term care for the elderly, and infant care. In addition to the aforementioned physiological information, the measurement of body temperature is also an important issue for patients, the elderly, and infants who need to keep a record of body temperature. Today, nursing manpower is not sufficient, and there is an urgent need to provide detection equipment for assisting in this care.

### BRIEF SUMMARY OF THE INVENTION

In an exemplary embodiment, the invention is directed to an FMCW (Frequency Modulated Continuous Wave) radar for detecting an object with a sensor. The FMCW radar includes a chirp signal generator, a transmitter module, a receiver module, a processing module, and a computing module. The chirp signal generator generates a radar signal. The transmitter module transmits the radar signal to the object. The receiver module receives a reflection signal from the object. The reflection signal includes a feature signal and a sense signal. The processing module generates an integrated digital signal according to the reflection signal and the radar signal. The computing module analyzes the integrated digital signal, so as to obtain first digital information and second digital information. The first digital information corresponds to the feature signal. The second digital information corresponds to the sense signal.

In an exemplary embodiment, the invention is directed to a detection method for an FMCW radar. The detection method includes the steps of: generating a radar signal by a chirp signal generator; transmitting the radar signal to an object, wherein the object has a sensor; receiving a reflection signal from the object, wherein the reflection signal includes a feature signal and a sense signal; generating an integrated digital signal according to the reflection signal and the radar signal; and analyzing the integrated digital signal, so as to obtain first digital information and second digital information, wherein the first digital information corresponds to the feature signal, and the second digital information corresponds to the sense signal.

### BRIEF DESCRIPTION OF DRAWINGS

The invention can be more fully understood by reading the subsequent detailed description and examples with references made to the accompanying drawings, wherein:

FIG. 1 is a diagram of an FMCW (Frequency Modulated Continuous Wave) radar according to an embodiment of the invention;

FIG. 2 is a diagram of a sensor according to an embodiment of the invention;

FIG. 3A is a diagram of an FMCW radar according to an embodiment of the invention;

FIG. 3B is a diagram of a computing module according to an embodiment of the invention;

FIG. 4 is a diagram of a waveform of an integrated digital signal according to an embodiment of the invention;

FIG. 5A is a diagram of a frequency variation of a radar signal according to an embodiment of the invention;

FIG. 5B is a diagram of a frequency variation of a radar signal according to an embodiment of the invention;

FIG. 5C is a diagram of a frequency-domain of an integrated digital signal after a range-FFT (Fast Fourier Transform) is performed, according to an embodiment of the invention;

FIG. 5D is a diagram of a time-domain of an integrated digital signal SI according to an embodiment of the invention;

FIG. 6 is a diagram of a time axis according to an embodiment of the invention;

FIG. 7 is a diagram of a time axis according to an embodiment of the invention; and

FIG. 8 is a flowchart of a detection method for an FMCW radar according to an embodiment of the invention.

### DETAILED DESCRIPTION OF THE INVENTION

In order to illustrate the purposes, features and advantages of the invention, the embodiments and figures of the invention are shown in detail as follows.

Certain terms are used throughout the description and following claims to refer to particular components. As one skilled in the art will appreciate, manufacturers may refer to a component by different names. This document does not intend to distinguish between components that differ in name but not function. In the following description and in the claims, the terms “include” and “comprise” are used in an open-ended fashion, and thus should be interpreted to mean “include, but not limited to . . . ”. The term “substantially” means the value is within an acceptable error range. One skilled in the art can solve the technical problem within a predetermined error range and achieve the proposed technical performance. Also, the term “couple” is intended to mean either an indirect or direct electrical connection. Accordingly, if one device is coupled to another device, that connection may be through a direct electrical connection, or through an indirect electrical connection via other devices and connections.

The following disclosure provides many different embodiments, or examples, for implementing different features of the provided subject matter. Specific examples of components and arrangements are described below to simplify the present disclosure. These are, of course, merely examples and are not intended to be limiting. For example, the formation of a first feature over or on a second feature in the description that follows may include embodiments in which the first and second features are formed in direct contact, and may also include embodiments in which additional features may be formed between the first and second features, such that the first and second features may not be in direct contact. In addition, the present disclosure may repeat reference numerals and/or letters in the various examples. This repetition is for the purpose of simplicity and clarity and does not in itself dictate a relationship between the various embodiments and/or configurations discussed.

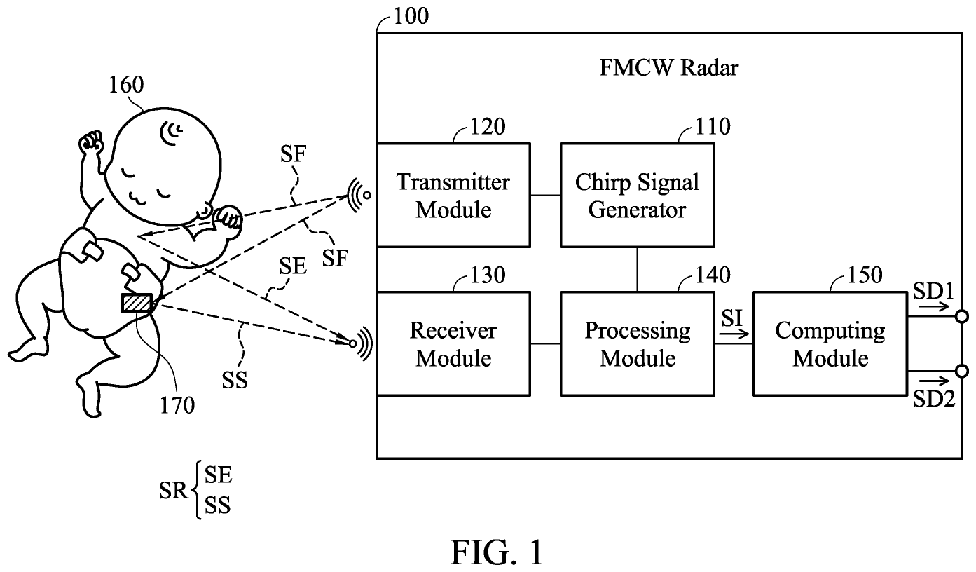

FIG. 1 is a diagram of an FMCW (Frequency Modulated Continuous Wave) radar 100 according to an embodiment of the invention. In the embodiment of FIG. 1, the FMCW radar 100 includes a chirp signal generator 110, transmitter module 120, a receiver module 130, a processing module 140, and a computing module 150. It should be understood that the FMCW radar 100 may further include other components, such as a housing, a controller, and/or a power supply module, although they are not displayed in FIG. 1.

In some embodiments, the FMCW radar 100 is configured to detect an object 160 with a sensor 170. It should be noted that both the object 160 and the sensor 170 are external elements, and they are not any portions of the FMCW radar 100. For example, the aforementioned object 160 may be a human body, and the sensor 170 may be disposed on/at any position of the aforementioned human body, but they are not limited thereto.

The chirp signal generator 110 is configured to generate a radar signal SF. In some embodiments, the radar signal SF has a linear modulation frequency. For example, the linear modulation frequency of the radar signal SF may be from 24 GHz to 26 GHz, or may be from 77 GHz to 81 GHz, but it is not limited thereto.

The transmitter module 120 is coupled to the chirp signal generator 110. The transmitter module 120 can transmit the radar signal SF to the object 160. In response, the receiver module 130 can receive a reflection signal SR from the object 160. The reflection signal SR includes a feature signal SE and a sense signal SS. Specifically, the feature signal SE may include the information related to the object 160, and the sense signal SS may include the information related to the sensor 170. For example, when the object 160 receives the radar signal SF, the object 160 can transmit the corresponding feature signal SE back. In some embodiments, the feature signal SE is a physiological signal.

The processing module 140 is coupled to the chirp signal generator 110 and the receiver module 130. The processing module 140 can generate an integrated digital signal SI according to the reflection signal SR and the radar signal SF. The computing module 150 is coupled to the processing module 140. The computing module 150 can analyze the integrated digital signal SI, so as to obtain first digital information SD1 and second digital information SD2. It should be noted that the first digital information SD1 corresponds to the feature signal SE, and the second digital information SD2 corresponds to the sense signal SS.

Specifically, the computing module 150 can perform a first signal processing procedure on the integrated digital signal SI, so as to obtain the first digital information SD1. In some embodiments, the first signal processing procedure includes a variety of algorithms, such as time-domain processing, frequency-domain processing, or AI (Artificial Intelligence) machine learning. The first digital information SD1, extracted from the integrated digital signal SI, may include the information related to breath and/or heartbeat, but it is not limited thereto.

In some embodiments, the aforementioned AI algorithms include supervised learning, un-supervised learning, semi-supervised learning, or reinforcement learning.

Furthermore, the computing module 150 can perform a second signal processing procedure on the integrated digital signal SI, so as to obtain the second digital information SD2. In some embodiments, the second signal processing procedure includes a variety of algorithms, such as time-domain processing or AI machine learning. The second digital information SD2, extracted from the integrated digital signal SI, may include the information related to temperature, humidity, pressure, and/or chemical composition, but it is not limited thereto.

Generally, the chirp signal generator 110 is used to increase the detection accuracy of the FMCW radar 100. In addition, the FMCW radar 100 merely uses the same transmitter module 120 and the same receiver module 130 to obtain two different types of information, and it effectively simplifies the overall circuit complexity and reduces the relative manufacturing cost. With such a design of the invention, the computing module 150 of the FMCW radar 100 can also separate the two different types of information, so as to improve the following analysis and process.

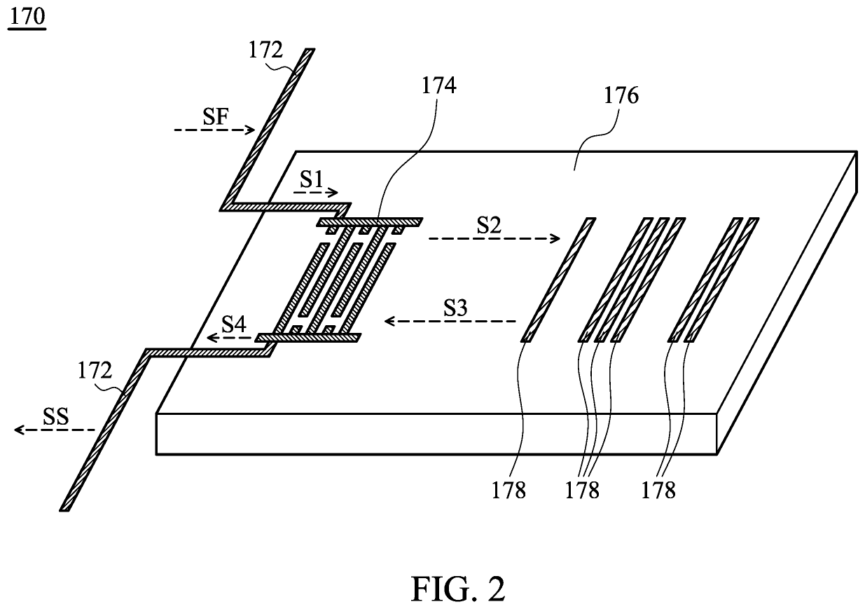

FIG. 2 is a diagram of the sensor 170 according to an embodiment of the invention. In the embodiment of FIG. 2, the sensor 170 is a passive SAW (Surface Acoustic Wave) sensor which includes an antenna module 172, an IDT (Interdigital Transducer) 174, a piezoelectric substrate 176, and one or more reflectors 178. For example, when the sensor 170 receives the radar signal SF, it can transmit the corresponding sense signal SS back.

In the beginning, the antenna module 172 can receive the radar signal SF, and generate a first electric signal S1 according to the radar signal SF. The IDT 174 is coupled to the antenna module 172, and is disposed on the piezoelectric substrate 176. Because of the reverse piezoelectric effect of the piezoelectric substrate 176, the IDT 174 can convert the first electric signal S1 into a first SAW signal S2. Next, the reflectors 178 can reflect the first SAW signal S2, so as to generate a second SAW signal S3. The second SAW signal S3 and the first SAW signal S2 may have exactly opposite directions of propagation. Because of the piezoelectric effect of the piezoelectric substrate 176, the IDT 174 can convert the second SAW signal S3 into a second electric signal S4. Finally, the antenna module 172 can receive the second electric signal S4, and may generate and transmit the aforementioned sense signal SS to the FMCW radar 100 according to the second electric signal S4.

It should be noted that the speed of propagation of the radar signal SF and the reflection signal SR is almost equal to the speed of light. However, the speed of propagation of the first SAW signal S2 and the second SAW signal S3 is only substantially equal to the speed of sound. The speed of sound is also affected by a surrounding temperature. Thus, there may be a relatively long delay time between the sense signal SS and the radar signal SF. In addition, because the feature signal SE has nothing to do with any SAW signals, the reception time point of the feature signal SE is usually earlier than the reception time point of the sense signal SS. In some embodiments, the computing module 150 of the FMCW radar 100 can estimate a possible temperature of the sensor 170 by analyzing the delay time of the sense signal SS. The possible temperature of the sensor 170 may be substantially the same as the current temperature of the object 160. In some embodiments, if the sensor 170 includes more reflectors 178, they can provide a plurality of delay times, so as to improve the accuracy of the aforementioned temperature estimation.

The following embodiments will introduce different configurations and detail structural features of the FMCW radar 100. It should be understood that these figures and descriptions are merely exemplary, rather than limitations of the invention.

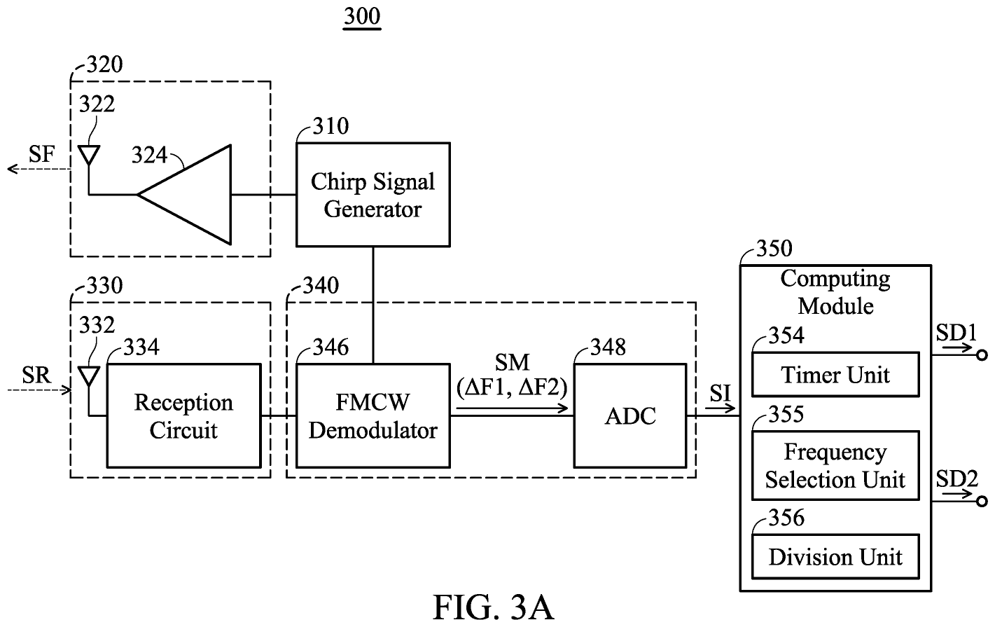

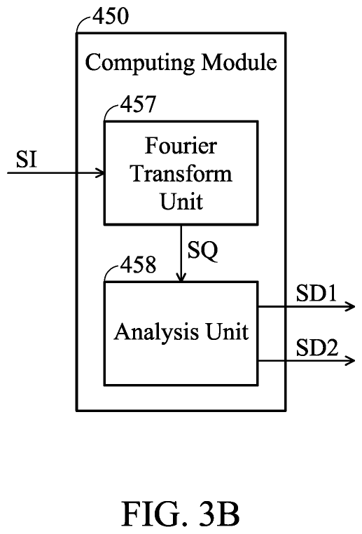

FIG. 3A is a diagram of an FMCW radar 300 according to an embodiment of the invention. FIG. 3A is similar to FIG. 1. In the embodiment of FIG. 3A, the FMCW radar 300 includes a chirp signal generator 310, a transmitter module 320, a receiver module 330, a processing module 340, and a computing module 350.

The chirp signal generator 310 can generate a radar signal SF. The transmitter module 320 includes a transmission antenna 322 and a power amplifier 324. The transmission antenna 322 is coupled through the power amplifier 324 to the chirp signal generator 310. Specifically, the power amplifier 324 can amplify the radar signal SF, and the transmission antenna 322 can transmit the amplified radar signal SF. The receiver module 330 includes a reception antenna 332 and a reception circuit 334. The reception circuit 334 is coupled to the reception antenna 332. The reception antenna 332 can receive a reflection signal SR. The reflection signal SR may include a feature signal SE and a sense signal SS. For example, the reception circuit 334 may include an LNA (Low Noise Amplifier) and a filter circuit, but it is not limited thereto.

As mentioned above, there is usually a relatively long delay time between the sense signal SS and the radar signal SF. Thus, in some embodiments, after the reception antenna 332 of the receiver module 330 receives the feature signal SE, the reception antenna 332 of the receiver module 330 can receive the sense signal SS.

The shapes and types of the transmission antenna 322 and the reception antenna 332 are not limited in the invention. In some embodiments, any of the transmission antenna 322 and the reception antenna 332 is a patch antenna, a monopole antenna, a dipole antenna, a loop antenna, a PIFA (Planar Inverted F Antenna), or a chip antenna.

The processing module 340 includes an FMCW demodulator 346 and an ADC (Analog-to-Digital Converter) 348. The FMCW demodulator 346 is coupled to the chirp signal generator 310 and the reception circuit 334, so as to obtain the radar signal SF and the reflection signal SR. For example, the FMCW demodulator 346 may include a mixer and a BPF (Band-Pass Filter), but it is not limited thereto. Next, the FMCW demodulator 346 can generate an IF (Intermediate Frequency) signal SM according to the reflection signal SR and the radar signal SF. In some embodiments, the IF signal SM includes the first frequency difference ΔF1 between the feature signal SE and the radar signal SF, and the second frequency difference ΔF2 between the sense signal SS and the radar signal SF. The ADC 348 is coupled to the FMCW demodulator 346. The ADC 348 can convert the IF signal SM into an integrated digital signal SI. In some embodiments, the FMCW demodulator 346 is integrated with the ADC 348, so as to form a single circuit.

The computing module 350 is coupled to the processing module 340. The integrated digital signal SI, which is received by the computing module 350, includes first digital information SD1 and second digital information SD2. In some embodiments, the computing module 350 includes a timer unit 354, a frequency selection unit 355, and a division unit 356, whose functions will be described in detail over the following embodiments. It should be understood that the timer unit 354, the frequency selection unit 355, and the division unit 356 may be implemented with hardware circuits or software programs. Furthermore, in some embodiments, the computing module 350 merely includes either the timer unit 354 or the frequency selection unit 355.

FIG. 3B is a diagram of a computing module 450 according to an embodiment of the invention. In the embodiment of FIG. 3B, the computing module 450 includes a Fourier transform unit 457 and an analysis unit 458. For example, the integrated digital signal SI may include a data matrix, and the Fourier transform unit 457 may have the function of performing an FFT (Fast Fourier Transform). Initially, the Fourier transform unit 457 can perform a range-FFT and a Doppler-FFT on the data matrix of the integrated digital signal SI, so as to generate a conversion result SQ. Next, the analysis unit 458 can analyze the conversion result SQ, so as to obtain the first digital information SD1 and the second digital information SD2 as mentioned above.

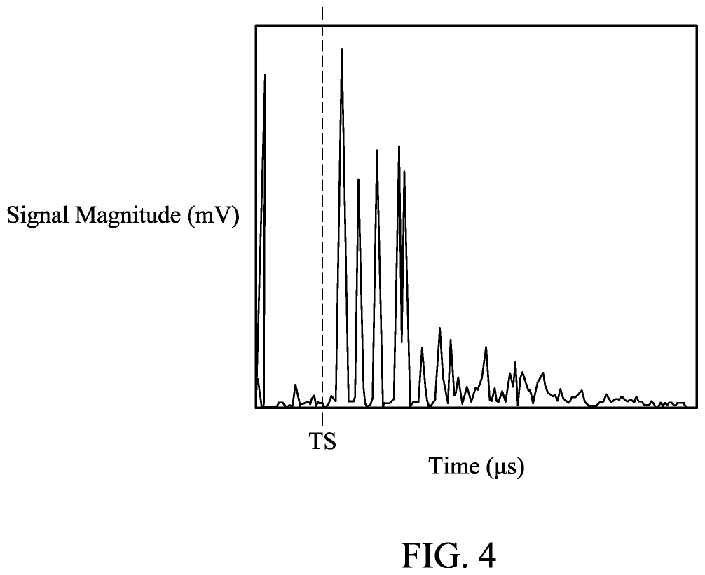

FIG. 4 is a diagram of a waveform of the integrated digital signal SI according to an embodiment of the invention. The horizontal axis represents time (μs), and the vertical axis represents signal magnitude (mV). As mentioned above, the first digital information SD1 corresponds to the feature signal SE, and the second digital information SD2 corresponds to the sense signal SS. In the embodiment of FIG. 4, since the reception time point of the sense signal SS is usually later than the reception time point of the feature signal SE, the computing module 350 can determine the integrated digital signal SI into the first digital information SD1 and the second digital information SD2 according to the partition time point TS.

Specifically, the first digital information SD1 may be before the partition time point TS, and the second digital information SD2 may be after the partition time point TS. Initially, the timer unit 354 of the computing module 350 can calculate the accurate partition time point TS. Next, the division unit 356 of the computing module 350 can use the partition time point TS to determine the integrated digital signal SI into front and back portions, where the front portion is considered as the first digital information SD1, and the back portion is considered as the second digital information SD2. In some embodiments, the partition time point TS is about 0.5 μs or 1 μs after the transmitter module 320 transmits the radar signal SF. For example, if the transmitter module 320 transmits the radar signal SF exactly at 0 μs, the partition time point TS may be set at 0.5 μs or 1 μs. The possible range of the partition time point TS is calculated and obtained according to many experimental results, and it helps to improve the accuracy of signal determination of the computing module 350 of the FMCW radar 300.

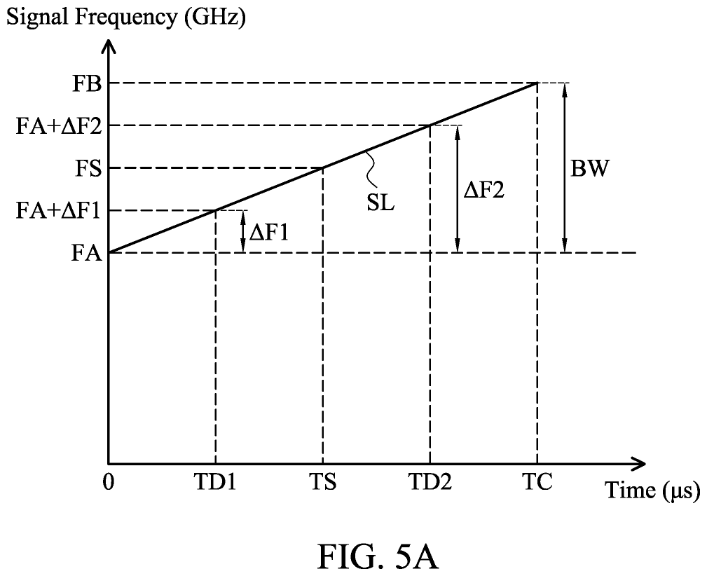

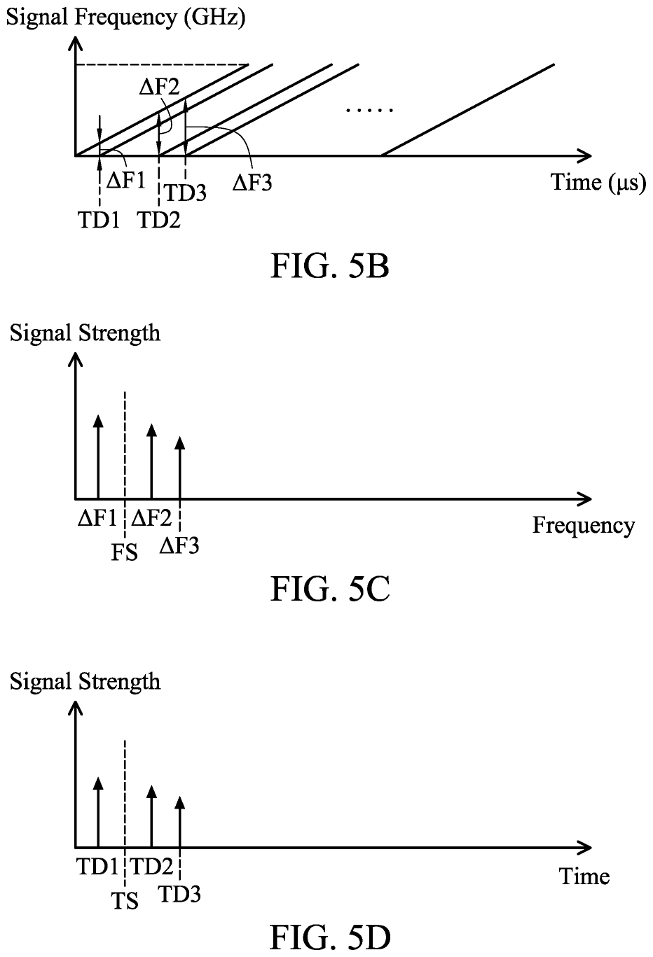

FIG. 5A is a diagram of a frequency variation of the radar signal SF according to an embodiment of the invention. The horizontal axis represents time (μs), and the vertical axis represents signal frequency (GHz). In the embodiment of FIG. 5A, the radar signal SF has a linear modulation frequency. In the beginning, the frequency of the radar signal SF is an initial frequency FA. Then, after a periodical time TC, the frequency of the radar signal SF rises to a final frequency FB. For example, the initial frequency FA may be 24 GHz or 77 GHz, the final frequency FB may be 26 GHz to 81 GHz, and the periodical time TC may be 40 μs, but they are not limited thereto. Specifically, the relative parameters of the radar signal SF will be described according to the following equations (1) and (2):

$$BW = FB - FA \qquad ( 1 )$$

$$SL = \frac{BW}{TC} \qquad ( 2 )$$

where “BW” represents an operational bandwidth BW of the radar signal SF, “SL” represents a rising slope SL of the radar signal SF, “FA” represents the initial frequency FA of the radar signal SF, “FB” represents the final frequency FB of the radar signal SF, and “TC” represents the periodical time TC of the radar signal SF.

According to the equation (1), the operational bandwidth BW of the radar signal SF can be calculated and obtained by subtracting the initial frequency FA from the final frequency FB. Also, according to the equation (2), the rising slope SL of the radar signal SF can be calculated and obtained by dividing the operational bandwidth BW by the periodical time TC.

For example, it is assumed that the transmitter module 320 transmits the radar signal SF exactly at 0 μs. The receiver module 330 may start to receive the feature signal SE after a first delay time TD1. Also, the receiver module 330 may start to receive the sense signal SS after a second delay time TD2. In comparison to the feature signal SE still having the initial frequency FA, the frequency of the corresponding radar signal SF may be equal to the initial frequency FA plus the first frequency difference ΔF1. Furthermore, in comparison to the sense signal SS still having the initial frequency FA, the frequency of the corresponding radar signal SF may be equal to the initial frequency FA plus the second frequency difference ΔF2. Specifically, the first delay time TD1 and the second delay time TD2 will be calculated and obtained according to the following equations (3) and (4):

$$TD 1 = \frac{Δ F 1}{SL} \qquad ( 3 )$$

$$TD 2 = \frac{Δ F 2}{SL} \qquad ( 4 )$$

where “TD1” represents the first delay time TD1, “TD2” represents the second delay time TD2, “ΔF1” represents the first frequency difference ΔF1, “ΔF2” represents the second frequency difference ΔF2, and “SL” represents the rising slope SL of the radar signal SF.

According to the equations (3) and (4), if the rising slope SL of the radar signal SF is known, the first delay time TD1 of the feature signal SE and the second delay time TD2 of the sense signal SS can be directly determined by analyzing the first frequency difference ΔF1 and the second frequency difference ΔF2. In some embodiments, the aforementioned partition time point TS is positioned between the first delay time TD1 and the second delay time TD2. Furthermore, at the partition time point TS, the corresponding frequency of the radar signal SF can be considered as the partition frequency point FS. Please note that in order to make it easier for readers to understand the relative relationship of the aforementioned parameters of the invention, FIG. 5A is not drawn according to the actual scale. In some embodiments, if a plurality of radar signals SF are used together, the computing module 450 can determine the movement speed or the phase variation of the object 160 by performing the Doppler-FFT.

In some embodiments, the computing module 350 can determine the integrated digital signal SI into the first digital information SD1 and the second digital information SD2 according to the partition frequency point FS. Specifically, the frequency of the feature signal SE may be lower than the partition frequency point FS, and the frequency of the sense signal SS may be higher than the partition frequency point FS. Initially, the frequency selection unit 355 of the computing module 350 can provide the accurate partition frequency point FS. Next, the division unit 356 of the computing module 350 can use the partition frequency point FS to determine the integrated digital signal SI into low and high portions, where the low portion is considered as the first digital information SD1, and the high portion is considered as the second digital information SD2. In some embodiments, the partition frequency point FS may be about 25 MHz or 50 MHz, which may correspond to 0.5 μs or 1 μs of the partition time point TS, respectively. The possible range of the partition frequency point FS is calculated and obtained according to many experimental results, and it helps to improve the accuracy of signal determination of the computing module 350 of the FMCW radar 300.

FIG. 5B is a diagram of a frequency variation of the radar signal SF according to an embodiment of the invention. In the embodiment of FIG. 5B, there are a plurality of reflection signals SR received at the first delay time TD1, the second delay time TD2, and the third delay time TD3, respectively. The first frequency difference ΔF1, the second frequency difference ΔF2, and the third frequency difference ΔF3 are formed between the corresponding radar signal SF and the aforementioned reflection signals SR. The IF signal SM may include information about the above frequency differences.

FIG. 5C is a diagram of a frequency-domain of the integrated digital signal SI after the range-FFT is performed, according to an embodiment of the invention. According to the measurement of FIG. 5C, there are relatively large signal strengths at the first frequency difference ΔF1, the second frequency difference ΔF2, and the third frequency difference ΔF3. By selecting a partition frequency point FS, a portion of the integrated digital signal SI which is lower than the partition frequency point FS includes information about the feature signal SE, and another portion of the integrated digital signal SI which is higher than the partition frequency point FS includes information about the sense signal SS, but the invention is not limited thereto.

FIG. 5D is a diagram of a time-domain of the integrated digital signal SI according to an embodiment of the invention. According to the equations (3) and (4), the integrated digital signal SI can be further converted into time-related signals. By selecting a partition time point TS, a portion of the integrated digital signal SI which is earlier than partition time point TS includes information about the feature signal SE, and another portion of the integrated digital signal SI which is later than the partition time point TS includes information about the sense signal SS, but the invention is not limited thereto.

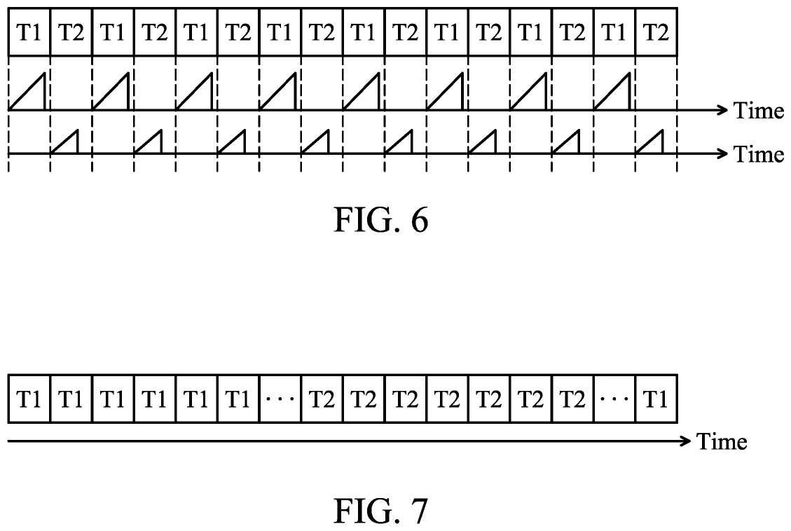

FIG. 6 is a diagram of a time axis according to an embodiment of the invention. As shown in FIG. 6, the time axis includes a plurality of first intervals T1 and a plurality of second intervals T2. The second intervals T2 are interleaved with the first intervals T1. In the embodiment of FIG. 6, the FMCW radar 100 can transmit the radar signal SF in each first interval T1, and then receive the feature signal SE from the object 160. As mentioned above, the first digital information SD1 can be determined by performing the first signal processing procedure, and the first digital information SD1 can correspond to the feature signal SE. In some embodiments, the first signal processing procedure includes a variety of algorithms, such as time-domain processing, frequency-domain processing, or AI machine learning. The first digital information SD1, extracted from the integrated digital signal SI, may include the information related to breath and/or heartbeat, but it is not limited thereto.

In addition, the FMCW radar 100 can also transmit the radar signal SF in each second interval T2, and then receive the sense signal SS from the sensor 170. As mentioned above, the second digital information SD2 can be determined by performing the second signal processing procedure, and the second digital information SD2 can correspond to the sense signal SS. In some embodiments, the second signal processing procedure includes a variety of algorithms, such as time-domain processing or AI machine learning. The second digital information SD2, extracted from the integrated digital signal SI, may include the information related to temperature, humidity, pressure, and/or chemical composition, but it is not limited thereto.

In some embodiments, the signal parameters of the radar signal SF in each second interval T2 are different from those of the radar signal SF in each first interval T1, such as different waveforms, different linear modulation frequencies, or different signal periods, but they are not limited thereto. In other words, it is not necessary to transmit the same radar signals in the first interval T1 and the second interval T2. According to practical measurements, such a design can further improve the detection accuracy within the first interval T1 and the second interval T2.

In conclusion, the FMCW radar 100 can use a design of time multiplexing to alternately receive, modulate, demodulate and compute the feature signal SE and the sense signal SS as mentioned above.

FIG. 7 is a diagram of a time axis according to an embodiment of the invention. FIG. 7 is similar to FIG. 6. As shown in FIG. 7, the time axis includes a plurality of first intervals T1 and a plurality of second intervals T2. The first intervals T1 can appear continuously, and the second intervals T2 can also appear continuously, but they are not limited thereto. Similarly, the FMCW radar 100 can also use another design of time multiplexing of FIG. 7 to alternately receive, modulate, demodulate and compute the feature signal SE and the sense signal SS as mentioned above.

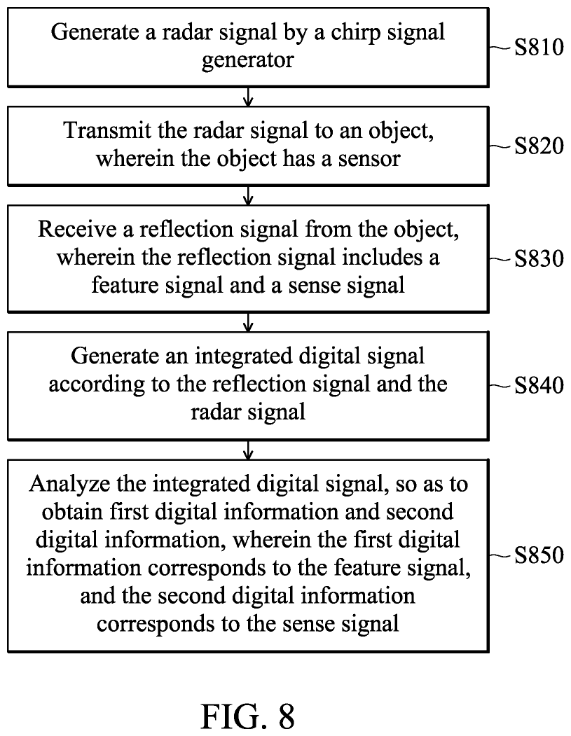

FIG. 8 is a flowchart of a detection method for an FMCW radar according to an embodiment of the invention. To begin, in step S810, a radar signal is generated by a chirp signal generator. In step S820, the radar signal is transmitted to an object. The object has a sensor. In step S830, a reflection signal is received from the object. The reflection signal includes a feature signal and a sense signal. In step S840, an integrated digital signal is generated according to the reflection signal and the radar signal. Finally, in step S850, the integrated digital signal is analyzed, so as to obtain first digital information and second digital information. The first digital information corresponds to the feature signal. The second digital information corresponds to the sense signal. It should be understood that these steps are not required to be performed in order, and every feature of the embodiments of FIGS. 1 to 7 may be applied to the detection method of FIG. 8.

The invention proposes a novel FMCW radar and a novel detection method. In comparison to the conventional design, the invention has at least the advantages of increasing the detection accuracy, simplifying the overall circuit complexity, and reducing the relative manufacturing cost. Therefore, the invention is suitable for application in a variety of devices.

Note that the above element parameters are not limitations of the invention. A designer can fine-tune these setting values according to different requirements. It should be understood that the FMCW radar and detection method of the invention are not limited to the configurations of FIGS. 1-8. The invention may include any one or more features of any one or more embodiments of FIGS. 1-8. In other words, not all of the features displayed in the figures should be implemented in the FMCW radar and detection method of the invention.

The method of the invention, or certain aspects or portions thereof, may take the form of program code (i.e., executable instructions) embodied in tangible media, such as floppy diskettes, CD-ROMS, hard drives, or any other machine-readable storage medium, wherein, when the program code is loaded into and executed by a machine such as a computer, the machine thereby becomes an apparatus for practicing the methods. The methods may also be embodied in the form of program code transmitted over some transmission medium, such as electrical wiring or cabling, through fiber optics, or via any other form of transmission, wherein, when the program code is received and loaded into and executed by a machine such as a computer, the machine becomes an apparatus for practicing the disclosed methods. When implemented on a general-purpose processor, the program code combines with the processor to provide a unique apparatus that operates analogously to application-specific logic circuits.

Use of ordinal terms such as “first”, “second”, “third”, etc., in the claims to modify a claim element does not by itself connote any priority, precedence, or order of one claim element over another or the temporal order in which acts of a method are performed, but are used merely as labels to distinguish one claim element having a certain name from another element having the same name (but for use of the ordinal term) to distinguish the claim elements.

It will be apparent to those skilled in the art that various modifications and variations can be made in the invention. It is intended that the standard and examples be considered as exemplary only, with a true scope of the disclosed embodiments being indicated by the following claims and their equivalents.

What is claimed is:

1. An FMCW (Frequency Modulated Continuous Wave) radar for detecting an object with a sensor, comprising:
a chirp signal generator, generating a radar signal;
a transmitter module, transmitting the radar signal to the object;
a receiver module, receiving a reflection signal from the object, wherein the reflection signal comprises a feature signal and a sense signal;
a processing module, generating an integrated digital signal according to the reflection signal and the radar signal; and
a computing module, analyzing the integrated digital signal, so as to obtain first digital information and second digital information;
wherein the first digital information corresponds to the feature signal, and the second digital information corresponds to the sense signal.

2. The FMCW radar as claimed in claim 1, wherein the sensor is an SAW (Surface Acoustic Wave) sensor.

3. The FMCW radar as claimed in claim 1, wherein the processing module comprises:
an FMCW demodulator, generating an IF (Intermediate Frequency) signal according to the reflection signal and the radar signal; and
an ADC (Analog-to-Digital Converter), converting the IF signal into the integrated digital signal.

4. The FMCW radar as claimed in claim 3, wherein the IF signal comprises a first frequency difference between the feature signal and the radar signal, and a second frequency difference between the sense signal and the radar signal.

5. The FMCW radar as claimed in claim 1, wherein the computing module comprises:
a Fourier transform unit, performing a range-FFT (Fast Fourier Transform) and a Doppler-FFT on the integrated digital signal, so as to generate a conversion result; and
an analysis unit, analyzing the conversion result, so as to obtain the first digital information and the second digital information.

6. The FMCW radar as claimed in claim 1, wherein the computing module determines the integrated digital signal into the first digital information and the second digital information according to a partition time point.

7. The FMCW radar as claimed in claim 1, wherein the computing module determines the integrated digital signal into the first digital information and the second digital information according to a partition frequency point.

8. The FMCW radar as claimed in claim 1, wherein the chirp signal generator provides the radar signal with different signal parameters in a first interval and a second interval.

9. The FMCW radar as claimed in claim 1, wherein the first digital information comprises information related to breath and/or heartbeat.

10. The FMCW radar as claimed in claim 1, wherein the second digital information comprises information related to temperature, humidity, pressure, and/or chemical composition.

11. A detection method for an FMCW radar, comprising:
generating a radar signal by a chirp signal generator;
transmitting the radar signal to an object, wherein the object has a sensor;
receiving a reflection signal from the object, wherein the reflection signal comprises a feature signal and a sense signal;
generating an integrated digital signal according to the reflection signal and the radar signal; and
analyzing the integrated digital signal, so as to obtain first digital information and second digital information, wherein the first digital information corresponds to the feature signal, and the second digital information corresponds to the sense signal.

12. The detection method as claimed in claim 11, wherein the sensor is an SAW sensor.

13. The detection method as claimed in claim 11, further comprising:
generating an IF signal according to the reflection signal and the radar signal by an FMCW demodulator; and
converting the IF signal into the integrated digital signal by an ADC.

14. The detection method as claimed in claim 13, wherein the IF signal comprises a first frequency difference between the feature signal and the radar signal, and a second frequency difference between the sense signal and the radar signal.

15. The detection method as claimed in claim 11, further comprising:
performing a range-FFT and a Doppler-FFT on the integrated digital signal, so as to generate a conversion result; and
analyzing the conversion result, so as to obtain the first digital information and the second digital information.

16. The detection method as claimed in claim 11, further comprising:
determining the integrated digital signal into the first digital information and the second digital information according to a partition time point.

17. The detection method as claimed in claim 11, further comprising:
determining the integrated digital signal into the first digital information and the second digital information according to a partition frequency point.

18. The detection method as claimed in claim 11, further comprising:
providing the radar signal with different signal parameters in a first interval and a second interval.

19. The detection method as claimed in claim 11, wherein the first digital information comprises information related to breath and/or heartbeat.

20. The detection method as claimed in claim 11, wherein the second digital information comprises information related to temperature, humidity, pressure, and/or chemical composition.
